(19) United States
(12) Patent Application Publication
(10) Pub. No.: US 2024/0175864 A1
(43) Pub. Date: May 30, 2024

(54) BIOLOGICAL TISSUE SAMPLE IMAGING METHOD

(71) Applicant: WESTLAKE UNIVERSITY, Zhejiang (CN)

(72) Inventors: Liang GAO, Zhejiang (CN); Ruili FENG, Zhejiang (CN); Yanlu CHEN, Zhejiang (CN)

(21) Appl. No.: 18/431,691

(22) Filed: Feb. 2, 2024

Related U.S. Application Data
(63) Continuation-in-part of application No. PCT/CN2022/079853, filed on Mar. 9, 2022.

(30) Foreign Application Priority Data
Aug. 2, 2021 (CN) 202110880199.1

(51) Int. Cl. G01N 33/52; G01N 1/30

(52) U.S. Cl. CPC G01N 33/52; G01N 1/30

(57) ABSTRACT

A biological tissue sample imaging method, comprising: (1) put a magnetic material plate, a gel precursor solution and a biological tissue sample in an embedding container, and then forming a gel to obtain a gel body, in which the biological tissue sample, and a porous plate having ferromagnetism are embedded; and (2) mount the gel body on the sample holder magnetic base of an imaging microscope, and image.

### CROSS-REFERENCE TO RELATED APPLICATION

This application is a continuation-in-part of International Application PCT/CN2022/079853, filed Mar. 9, 2022, and claims the benefit of Chinese Application CN 202110880199.1, filed Aug. 2, 2021, the contents of each are hereby incorporated in their entirety.

### TECHNICAL FIELD

The invention relates to the technical field of biological tissue sample imaging methods, in particular to a biological tissue sample imaging method including a gel embedding step.

### BACKGROUND ART

High-resolution 3D fluorescence imaging of a biological tissue is an effective means to obtain 3D structure of the biological tissue and study biological issues such as gene expression, cell morphology, and cell distribution at subcellular, cellular, and tissue levels.

Biological tissue clearing techniques make biological tissue transparent, so as to overcome the main obstacle of high-resolution 3D imaging of biological tissue using fluorescence microscopy, and cutting-edge 3D fluorescence microscopy imaging techniques, such as light sheet microscopy, can be used to efficiently obtain cellular and subcellular 3D structural information of various biological tissues, thereby helping researchers better understand the structure and function of biological tissues and organs. Due to this significant advantage, biological tissue clearing techniques have been quickly applied to various fields of life science research.

Among various biological tissue clearing techniques, for example, the CUBIC hydrophilic clearing method has gained much attention because of the advantages of high biological safety, high retention rate of endogenous fluorescent proteins, compatibility with immunostaining, suitability for high-resolution imaging, etc.

After the biological tissue sample is treated with a hydrophilic clearing method, most of the lipid molecules in the tissue are removed, and the mechanical strength of the biological tissue becomes lower, and the biological tissue becomes soft, easily deformed or broken, so that it is difficult for the biological tissue sample to maintain the integrity of the biological tissue in the subsequent imaging process, which causes great difficulties for the 3D imaging of the cleared biological tissue using an optical microscope.

### SUMMARY OF THE INVENTION

The inventors found that biological tissue samples can be protected to an extent by gel embedding cleared biological tissue samples with agarose or the like to solve the problem of inconvenient transfer. However, the gel formed by agarose or the like is prone to cracking and breaking during transfer. In addition, it is difficult to fix a gel-embedded biological sample on the sample holder of a microscope, and it is not easy to adjust the position of the sample after it is fixed on the holder. Therefore, it could significantly hinder the user experience and reduce the imaging quality.

In order to solve the above problems, the present invention provides a method for imaging a biological tissue sample, comprising the steps of:
(1) putting a magnetic material plate, a gel precursor solution and a biological tissue sample in an embedding container, and then forming a gel to obtain a gel body, in which the biological tissue sample and the porous plate having ferromagnetism magnetic material plate are embedded;
(2) mounting the gel body on a sample holder magnetic base of an imaging microscope, and imaging.

The biological tissue may be a biological tissue selected from brain, spinal cord, heart, liver, lung, spleen, kidney, and other organs.

The biological tissue sample may be the whole or a part of the above-mentioned tissue.

The tissue organism may be one or more selected from biological research model animals. The biological research model animal may be, for example, nematodes, zebrafish, planarians, fruit flies, Xenopus laevis, salamanders, mice, rabbits, pigs, monkeys, etc.

Alternatively, the tissue organism may be a vertebrate, including a mammal, a reptile, a bird, etc. The mammal may be, for example, a human, a mouse, a rabbit, a pig, a monkey, etc.

The magnetic material plate refers to a plate-form magnetic material. The magnetic material may be selected from hard and soft magnetic materials, such as ferromagnetic materials, ferrite materials, rare earth permanent magnetic materials and the like.

The plate may be selected from a rigid plate, a flexible plate, a porous plate and a non-porous plate.

In some embodiments, the biological tissue sample is a biological tissue sample that has been cleared using a hydrophilic or hydrogel-based clearing method.

The gel precursor solution is a solution of 1.5% to 3%, preferably 1.8% to 2.5%, especially about 2% by mass of agarose in a refractive index matching solution.

In some embodiments, in the obtained gel body, the magnetic material plate is at the bottom, and the biological tissue sample is located above the magnetic material plate.

### MODE FOR THE INVENTION

The present disclosure will be described in detail below with specific embodiments, to enable those skilled in the art to better understand the technical solution of the present disclosure. The embodiments of the present disclosure will be described in further detail below in conjunction with specific examples, but the present disclosure is not limited thereto.

“First”, “second” and similar words used in the present disclosure do not indicate any order, quantity or importance, but are only used to distinguish different parts. “Comprising” or “including” and similar words mean that the element preceding the word cover the elements listed after the word, and do not exclude the possibility of also covering other elements. “Above”, “below”, “left”, “right” and so on are only used to indicate the relative positional relationship, and when the absolute position of the described object changes, the relative positional relationship may also change accordingly.

In the present disclosure, when it is described that a specific device is located between a first device and a second device, there may or may not be an intervening device between the specific device and the first device or the second device. When it is described that a specific device is connected to other device, the specific device may be directly connected to the other device without an intervening device, or may not be directly connected to the other device but has an intervening device.

All terms (including technical terms or scientific terms) used in the present disclosure have the same meaning as understood by those skilled in the relevant art, unless otherwise specifically defined. It should also be understood that terms defined in, for example, general dictionaries should be interpreted as having meanings consistent with their meanings in the context of the relevant technology, and should not be interpreted in an idealized or extremely formalized sense, unless explicitly defined herein.

Techniques, methods and devices known to those skilled in the relevant art may not be discussed in detail, but where appropriate, such techniques, methods and devices should be considered as part of the specification.

The present invention provides a method for imaging a biological tissue sample, comprising the steps of:
(1) putting a magnetic material plate, a gel precursor solution and a biological tissue sample in an embedding container, and then forming a gel to obtain a gel body, in which the biological tissue sample and the magnetic material plate are embedded;
(2) mounting the gel body on a sample holder magnetic base of an imaging microscope, and imaging.

In the method according to the present invention, the biological tissue is not particularly limited, and may be any tissue from an animal, such as brain, spinal cord, heart, liver, lung, spleen, kidney, and other organs.

The biological tissue sample may be the whole or a part of the above-mentioned tissue.

The tissue organism may be one or more selected from biological research model animals. The biological research model animals may be, for example, nematodes, zebrafish, planarians, fruit flies, Xenopus laevis, salamanders, mice, rabbits, pigs, monkeys, etc.

Alternatively, the tissue organism may be a vertebrate, including a mammal, a reptile, a bird, etc. The mammal may be, for example, a human, a mouse, a rabbit, a pig, a monkey, etc.

In the above step (1), the embedding container is not particularly limited, and those skilled in the art can select a suitable existing container or mold, or design and prepare a suitable container or mold, according to the size, shape and the like of the biological tissue sample to be imaged.

The magnetic material plate refers to a plate-form magnetic material. The magnetic material is not particularly limited, as long as it is a material that can be attracted by a permanent magnetic material (such as a magnet) or an electromagnet. For example, it may be a hard or soft magnetic material, such as a ferromagnetic material, a ferrite material, a rare earth permanent magnetic material, etc., such as iron, cobalt, nickel, gadolinium, iron-carbon alloy, iron-nickel alloy, iron-cobalt-nickel alloy, iron-cobalt alloy, iron-aluminum alloy, iron-silicon alloy, iron-silicon-aluminum alloy, iron-nickel-manganese alloy, an alloy material of iron and rare earth element, or a ferrite material, etc. In addition, the plate may be a plate formed entirely of a magnetic material, such as an iron plate, a silicon steel plate, a nickel plate, etc., or a plate formed of a non-magnetic material and a magnetic material, such as a plate formed by coating a magnetic material on the surface of a non-magnetic material plate, or a plate formed by coating a non-magnetic material on the surface of a magnetic material plate, but the present invention is not limited thereto. In addition, the plate may be a rigid plate, or a flexible plate, and may be a porous plate or a non-porous plate, but the present invention is not limited thereto.

By incorporating a magnetic material plate during formation of the gel body, it is more convenient to mount the gel body on a sample holder magnetic base, and also to adjust the gel body position after mounting, which reduce the occurrence of cracking and breaking of the gel body during sample mounting and position adjustment.

The gel precursor solution refers to a solution capable of forming a gel through gelation, and may be prepared by dissolving any suitable gelling reagent in a solvent.

In some embodiments, the gelling reagent may be agarose, poly-lysine, collagen, gelatin, polyacrylamide, etc., but the present invention is not limited thereto, preferably the gelling reagent is agarose. At this time, gelation may be achieved, for example, by dissolving the gelling reagent in a solvent at a high temperature and then cooling.

The solvent may be water, or a solution prepared for a certain purpose, such as a refractive index matching solution used in a hydrophilic or hydrogel-based clearing method.

In some embodiments, the biological tissue sample is a biological tissue sample that has been cleared by a hydrophilic or hydrogel-based clearing method, and the gel precursor solution is a solution of 1.5% to 3%, preferably 1.8% to 2.5%, especially about 2% by mass of agarose in a refractive index matching solution.

The clearing of the biological tissue sample includes delipidation and refractive index matching. The delipidation and refractive index matching may be performed by using any suitable delipidation reagent and refractive index matching reagent used in any hydrophilic or hydrogel-based clearing method in the art. For example, the delipidation reagent may be a CUBIC or CUBIC-L delipidation reagent, and the refractive index matching reagent may be a CUBIC-R refractive index matching reagent, but the present invention is not limited thereto. Specific delipidation reagents and refractive index matching reagents may be obtained by referring to papers or books related to hydrophilic or hydrogel-based clearing methods.

In some embodiments, in the obtained gel body, the magnetic material plate is at the bottom, and the biological tissue sample is located above the magnetic material plate.

In step (2), the magnetic base means that the base has a magnetic component, which has the magnetism to attract the above-mentioned magnetic material plate. The device may be permanently magnetic or electromagnetic. The magnetic base is usually made from a chemically stable material, such as stainless steel or Teflon, and is embedded or attached in another manner with a magnetic material for attracting the gel body embedded with the magnetic material plate. The shape of the magnetic base can be designed in different ways according to the characteristics and imaging methods of the corresponding microscope. After the embedded gel body is attracted on the magnetic base, the magnetic base can be mounted on an optical microscope for imaging the embedded sample, thus allowing 3D imaging of the cleared and embedded biological tissue.

By embedding a ferromagnetic plate in the gel body and using a magnetic base on the sample holder, the gel-embedded sample can be mounted on the sample holder magnetic base of the imaging microscope by magnetic force, so that it is easier to adjust the sample position and perform the 3D imaging, thereby solving the problems that the gel-embedded biological sample is not easy to be mounted on the conventional sample holder of imaging microscopes in which it is difficult to adjust the sample position, thus it is difficult to optimize the imaging quality.

Herein, all characteristics or conditions defined in the form of numerical or percentage ranges are only for the sake of simplicity and convenience. Accordingly, the description of a numerical or percentage range should be deemed to encompass and specifically disclose all possible sub-ranges and individual values within the range.

Herein, under the premise that the object of the invention can be achieved, the numerical value should be understood as having the precision of the effective digit of the numerical value. For example, the number 40.0 should be understood to cover the range from 39.50 to 40.49. Except for the working examples provided at the end of the detailed description, all values of parameters (for example, amounts or conditions) in this specification (including the appended claims) should in all cases be understood as being modified by the term “about”, regardless of whether “about” actually precedes the value. “About” indicates that the stated value allows for some imprecision (some close to exactness in the value; approximately or reasonably close to the value; approximate). If the imprecision provided by “about” is not understood in the art with this ordinary meaning, then “about” as used herein at least indicates the variation that can be produced by ordinary methods of measuring and using these parameters. For example, “about” can include variations of 10% or less, 5% or less, 4% or less, 3% or less, 2% or less, 1% or less, or 0.5% or less, and in some respects, a variation of less than or equal to 0.1%.

Furthermore, while exemplary embodiments have been described herein, the scope includes any and all embodiments having equivalent elements, modifications, omissions, combinations (e.g., technical solutions of combining various embodiments), adaptations or variations based on the present disclosure. Elements in the claims are to be interpreted broadly based on the language employed in the claims and are not limited to embodiments described in this specification or during the practice of the application, which embodiments are to be construed as non-exclusive. It is therefore intended that the specification and embodiments should be considered as being illustrative only, and the true scope and spirit are indicated by the full scope of the following claims and their equivalents.

The above description is intended to be illustrative rather than restrictive. For example, the above embodiments (or one or more technical solutions thereof) may be used in combination with each other. For example, other embodiments may be used by those skilled in the art upon reading the above description. Additionally, in the above specific embodiments, various features may be grouped together in order to simplify the disclosure. This is not to be interpreted as intending that an unclaimed disclosed feature is essential to any claim. Rather, the disclosed subject matter may lie in less than all features of a particular disclosed embodiment. Thus, the following claims are hereby incorporated into the detailed description as examples or embodiments, where each claim stands on its own as a separate embodiment, and it is contemplated that these embodiments may be combined with each other in various combinations or permutations. The scope of the present disclosure should be determined with reference to the full scope of the appended claims and the equivalents to which such claims are entitled.

The above embodiments are only exemplary embodiments of the present disclosure, and are not intended to limit the present disclosure, and the protection scope of the present disclosure is defined by the claims. Various modifications or equivalent replacements may be made by those skilled in the art to the present disclosure within the spirit and protective scope of the present disclosure, and such modifications or equivalent replacements shall also be deemed to fall within the protective scope of the present disclosure.

1. A method for imaging a biological tissue sample, comprising the steps of:
(1) putting a magnetic material plate, a gel precursor solution and a biological tissue sample in an embedding container, and then forming a gel to obtain a gel body, in which the biological tissue sample and the magnetic material plate are embedded;
(2) mounting the gel body on the sample holder magnetic base of an imaging microscope, and imaging.

2. The method according to claim 1, wherein the biological tissue is a biological tissue selected from brain, spinal cord, heart, liver, lung, spleen, kidney, and other organs.

3. The method according to claim 1, wherein the biological tissue sample is the whole or a part of the biological tissue.

4. The method according to claim 1, wherein the tissue organism is one or more selected from biological research model animals.

5. The method according to claim 1, wherein the tissue organism is a vertebrate.

6. The method according to claim 5, wherein the vertebrate includes a mammal, a reptile and a bird.

7. The method according to claim 6, wherein the mammal is a human, a mouse, a rabbit, a pig, or a monkey.

8. The method according to claim 1, wherein the magnetic material is selected from hard and soft magnetic materials.

9. The method according to claim 1, wherein the magnetic material is selected from ferromagnetic materials, ferrite materials, and rare earth permanent magnetic materials.

10. The method according to claim 1, wherein the plate is selected from a rigid plate, a flexible plate, a porous plate and a non-porous plate.

11. The method according to claim 1, wherein the biological tissue sample is a biological tissue sample that has been cleared using a hydrophilic or hydrogel-based clearing method.

12. The method according to claim 1, wherein the gel precursor solution is a solution of 1.5% to 3% by mass of agarose in a refractive index matching solution.

13. The method according to claim 1, wherein the gel precursor solution is a solution of 1.8% to 2.5% by mass of agarose in a refractive index matching solution.

14. The method according to claim 1, wherein the gel precursor solution is a solution of 2% by mass of agarose in a refractive index matching solution.

15. The method according to claim 1, wherein, in the obtained gel body, the magnetic material plate is at the bottom, and the biological tissue sample is located above the magnetic material plate.
